(19) United States
(12) Patent Application Publication
(10) Pub. No.: US 2006/0165571 A1
(43) Pub. Date: Jul. 27, 2006

(54) Nipple overcap having sterilizer

(76) Inventor: Kim Sang Seon, Bupyung-gu Incheon (KR)

Correspondence Address: BELL, BOYD & LLOYD, LLC, PO BOX 1135, CHICAGO, IL, 60690-1135, US

(21) Appl. No.: 11/264,897

(22) Filed: Nov. 1, 2005

(30) Foreign Application Priority Data
Jun. 29, 2005 (KR) 2005-56709
May 6, 2005 (KR) 2005-12686
May 6, 2005 (KR) 2005-12687
Jan. 24, 2005 (KR) 2005-2162

(51) Int. Cl. A61L 2/20

(52) U.S. Cl. 422/302; 422/305; 422/24; 422/186.07

(57) ABSTRACT

Disclosed herein is a nipple overcap having a sterilizer including: a nursing bottle overcap detachably mounted on a nipple-fixing cap having a nipple and having a through-hole formed at the center thereof, a part of the upper portion of the nipple being located on the nursing bottle overcap; a substrate case detachably mounted on the upper portion of the nursing bottle overcap and having a sound generator; and an ozone generator mounted between the substrate case and the nursing bottle overcap, wherein the ozone generator includes: a rechargeable battery for supplying power source to the sound generator and the ozone generator; an electric circuit board for executing a preset program using power source supplied from the rechargeable battery; and an ozone lamp connected to the electric circuit board. The nipple overcap can sterilize various bacteria parasitic on the nipple by the ozone generator, and generate sounds according to the operation state of the sterilizer by the sound generator.

### CROSS REFERENCE TO RELATED APPLICATION

This application claims priority to and the benefit of Korean Utility Model Application Nos. 20-2005-12686, 20-2005-12687, 20-2005-2162, and Korean Patent Application No. 10-2005-56709 filed in the Korean Intellectual Property Office on May 6, 2005, May 6, 2005, Jan. 24, 2005 and Jun. 29, 2005, the entire content of which is incorporated herein by reference.

### BACKGROUND

The present invention relates to a nipple overcap having a sterilizer, and more particularly, to a nipple overcap having a sterilizer, which can remove bacteria on a nipple by sterilizing the nipple with ozone and ultraviolet ray.

A nursing bottle includes a sealed body, and a nipple-fixing cap coupled with the nipple. Milk is contained inside the body of the nursing bottle, and the body of the nursing bottle has a spiral portion formed on the upper end portion. The nipple-fixing cap is coupled with the spiral portion, and includes the nipple made of silicon and having a fine hole formed at the center thereof. Furthermore, the nipple-fixing cap has a coupling portion which is coupled with an overcap for preventing pollution of the nipple.

Such nursing bottle is to treating a baby with milk in lieu of mother's milk, and used several times a day. Particularly, since the baby cannot drink milk contained in the nursing bottle at once, the baby drinks milk over and over. In addition, if the baby does not drink milk, a user covers the nipple with the overcap to keep the contained milk safely for some time.

However, when the user lets the baby put the nipple in and out the mouth, the baby's spittle is stained on the nipple, and so, bacteria in the air propagates on the nipple by the baby's spittle. Therefore, if the baby puts the nipple in the mouth, the baby may be infected with bacteria. Therefore, the user must not reuse the used nursing bottle as it is but reuse it after sterilizing in hot water.

To sterilize the nipple of the nursing bottle, the nipple is sterilized through a process of putting water and the nipple in a sterilizing tank and heating and boiling water for several tens minutes. However, such sterilizing process is very inconvenient and requires much time. When the user is busy, the user sometimes uses the nipple without, and thereby, the baby is sometimes infected with bacteria. Moreover, the conventional sterilizing process has another problem in that the user cannot check whether or not the sterilization of the nipple is completed.

In addition, the user cannot sterilize the nursing bottle when the user goes out with the baby, and the baby is infected with bacteria when the baby puts the nipple in the mouth. The user has to prepare a number of nursing bottles to solve the above problems, and cannot reuse the nursing bottle even though milk remains in the nursing bottle.

The information disclosed in this Background of the Invention section is only for enhancement of understanding of the background of the invention, and therefore, unless explicitly described to the contrary, it should not be taken as an acknowledgement or any form of suggestion that this information forms the prior art that is already known in this country to a person of ordinary skill in the art.

### SUMMARY

Therefore, the present invention has been made for solving the above problems occurring in the prior art, and it is an object of the present invention to provide a nipple overcap having a sterilizer, which can be detachably mounted on a nipple coupled with a nursing bottle.

Another object of the present invention is to provide a nipple overcap having a sterilizer, which includes a solar cell and a sound generator, so that the nipple overcap is rechargeable without exchanging a battery, and the sterilizer can inform a progress state, i.e., start, progress and end of sterilization.

To accomplish the above objects, there is provided a nipple overcap having a sterilizer according to the present invention. The nipple overcap includes a nursing bottle overcap for detachably mounting on a nipple-fixing cap having a nipple and having a through-hole formed at the center thereof, for a part of the upper portion of the nipple; a substrate case for detachably mounting on the top of the nursing bottle overcap, which have a sound generator; and an ozone generator being located between the substrate case and the nursing bottle overcap, wherein the ozone generator includes: a rechargeable battery for supplying power to the sound generator and the ozone generator; an electric circuit board for performing a preset program using power source supplied from the rechargeable battery; and an ozone lamp connected to the electric circuit board.

The substrate case includes a solar cell mounted on the upper surface thereof for absorbing light and converting the light into electric energy to charge the rechargeable battery.

The sound generator is connected with the rechargeable battery, and includes: a memory for storing melodies or sounds; and a speaker for transmitting melodies or sounds generated from the memory to the outside, wherein the speaker is mounted on the substrate case.

The substrate case includes: a timer circuit part for setting an operation time period of the ozone lamp; and an ozone generation control circuit part for uniformly controlling generation of ozone by the ozone lamp.

The substrate case further includes an LED display for indicating a progress state, i.e., start, progress and end of sterilization.

The substrate case further includes an LCD display for indicating the progress state of sterilization with characters or numbers.

The electric circuit board further includes light sensitivity controlling part for controlling brightness of the ozone lamp.

Additional features and advantages are described herein, and will be apparent from, the following Detailed Description and the figures.

### BRIEF DESCRIPTION OF THE FIGURES

The accompanying drawings illustrate exemplary embodiments of the present invention, and, together with the description, serve to explain the principles of the present invention, wherein:

The above and other objects, features and advantages of the present invention will be apparent from the following detailed description of the preferred embodiments of the invention in conjunction with the accompanying drawings, in which:

FIG. 1 is an exploded perspective view of a nipple overcap having a sterilizer and a nursing bottle according to the present invention;

FIG. 2 is an exploded perspective view of the nipple overcap having the sterilizer according to the present invention;

FIG. 3 is a bottom perspective view of an electric circuit board mounted in the nipple overcap having the sterilizer according to the present invention; and

FIG. 4 is a block diagram showing a structure of the electric circuit board of the nipple overcap having the sterilizer according to the present invention.

### DETAILED DESCRIPTION

In the following detailed description, only certain exemplary embodiment of the present invention has been shown and described, simply by way of illustration. As those skilled in the art would realize, the described embodiments may be modified in various different ways, all without departing from the spirit or scope of the present invention. Accordingly, the drawings and description are to be regarded as illustrative in nature, and not restrictive.

An embodiment of the present invention will hereinafter be described in detail with reference to the accompanying drawings.

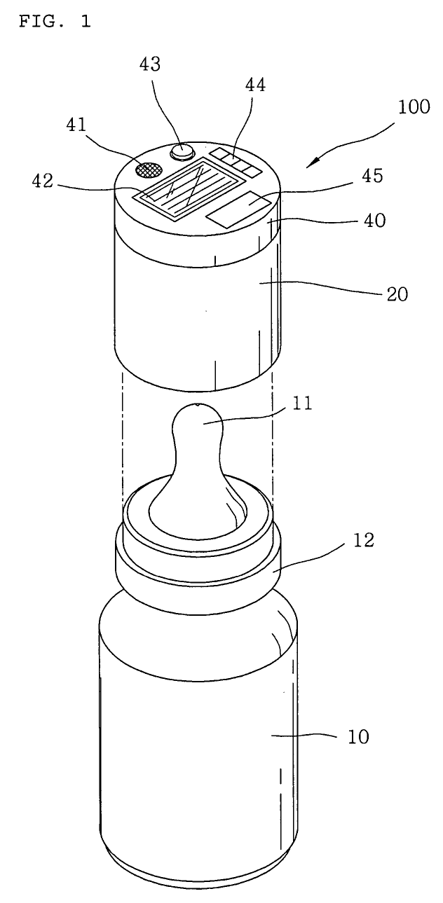

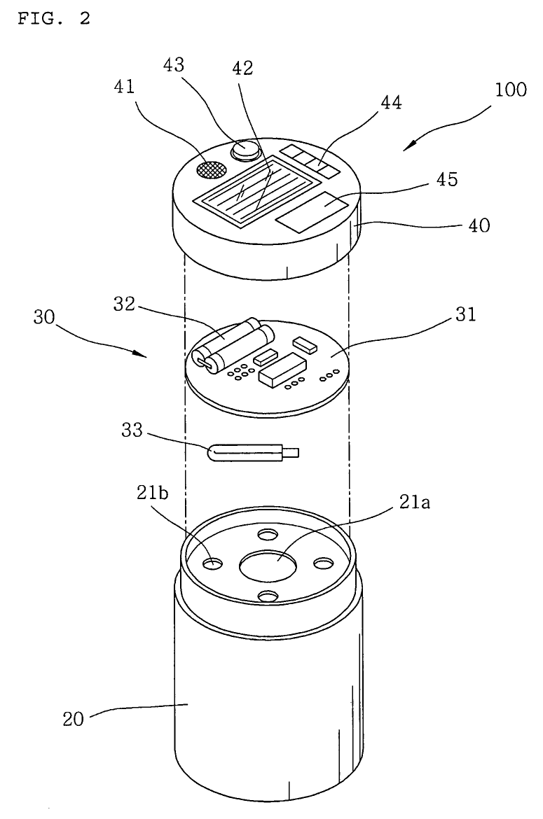

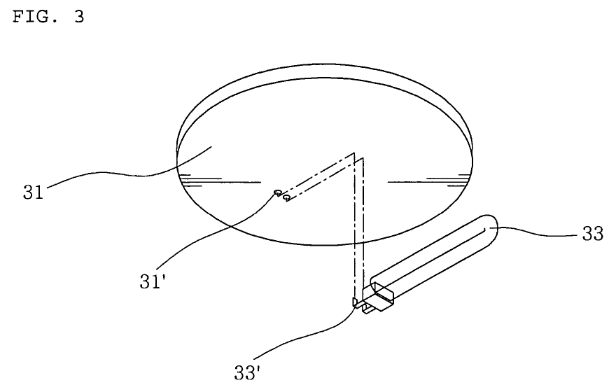

As shown in FIGS. 1 to 3, a nipple overcap having a sterilizer according to an exemplary embodiment of the present invention includes an overcap 20, an ozone generator 30 and a substrate case 40.

The nursing overcap 20 is to cover a nipple part 11 of a nipple-fixing cap 12 detachably mounted on the nursing bottle 10, and is generally in the form of a cylinder in its outward shape. The nursing bottle overcap 20 includes a partition 21 formed on the upper portion thereof. The partition 21 divides the nursing bottle overcap 20 into the lower space and the upper space, and has a through-hole 21a formed at the center thereof. Further the partition 21 can have a number of holes 21b equiangularly and radially formed from the through-hole 21a. Furthermore, the nursing bottle overcap 20 includes a holding protrusion formed on the inner side wall of the lower end thereof, and the nipple-fixing cap 12 has a holding groove formed correspondingly to the holding protrusion.

The ozone generator 30 is used for sterilizing the nipple part 11, and includes an electric circuit board 31, a rechargeable battery 32, and an ozone lamp 33.

The electric circuit board 31 is established in the upper space of the nursing bottle overcap 20. The electric circuit board 31 has a socket 31′, and the ozone lamp 33 has a plug 33′ which can be coupled with the socket 31′. By the above structure, the ozone lamp 33 can be inserted with the electric circuit board 31. To wash the nursing bottle overcap 20, when the electric circuit board 31 is separated from the upper space of the nursing bottle overcap 20, the ozone lamp 33 is also separated with the electric circuit board 31.

Moreover, the electric circuit board 31 includes a timer circuit part 31a for generating a signal for setting an operation period of the ozone lamp 33, a sound circuit part 31b for generating a melody signal or a sound signal when sterilization by the ozone lamp 33 is in progress or completed, an ozone generation control circuit part 31c for uniformly controlling ozone generation by the ozone lamp 33, and a controller 34 for controlling the timer circuit part 31a, the sound circuit part 31b and the ozone generation control circuit part 31c. Here the operation time of the ozone lamp 33 is set by the timer circuit part 31a. For example, the ozone lamp 33 can be operated in 10 minutes, 20 minutes or 30 minutes. Additionally, the electric circuit board 31 further includes a light sensitivity controlling part (not shown) for controlling brightness of ultraviolet ray generated by the ozone lamp 33. The light sensitivity controlling part is connected with the controller 34, and so, automatically control light generated from the ozone lamp 33 on the base of the surrounding brightness and the operation time of the ozone lamp 33.

Moreover, the sound circuit part 31b transmits a melody signal or a sound signal to a speaker 41 which will be described later, and includes a memory for storing melodies or sounds. Furthermore, the ozone generation controlling part 31c is connected with a CDS (light sensitivity controlling device; 31c′) established at a predetermined portion of the nursing bottle overcap 20. The CDS 31c′ measures the strength of ultraviolet ray generated from the ozone lamp 33 and transmits a signal corresponding to the ozone generation control circuit part 31c. The ozone generation control circuit part 31c uniformly controls the ozone generated by the ozone lamp 33 in response to the signal transmitted from the CDS 31c″.

Meanwhile, the rechargeable battery 32 charges electricity generated from a solar cell 42 which will be described later. Therefore, the nipple overcap according to the exemplary embodiment of the present invention can be provided with power source without additional charging device. A general small-sized battery can be used as the rechargeable battery 32.

It is preferable that the ozone lamp 33 generates 253.7 nm wave of ultraviolet ray for good sterilization property and 185 nm wave of ultraviolet ray for generating ozone. That is, the 253.7 nm wave of ultraviolet ray directly sterilizes bacteria, and the 185 nm wave of ultraviolet ray converts oxygen into ozone for sterilizes the bacteria, so that the bacteria can be clearly removed on the nipple 11. The substrate case 40 includes the speaker 41, a switch 42, the solar cell 43, an LED display 44, and an LCD display 45 disposed on the upper surface thereof.

The solar cell 42 absorbs light from the indoor light of home or the sun and converts the absorbed light into electric energy. The electric energy automatically is charged into the rechargeable battery 32 for operation of the ozone lamp 33. The switch 43 is an ON/OFF switch 43 for operating the sterilizer 100. The speaker 41 generates various sounds to the outside when the sterilizer 100 is operated. The substrate case 40 has the LED display 44 for display light signal corresponding to the progressed state of the sterilizer, and the LCD display 45 for informing the progressed state of the sterilization with characters and numbers.

Also, the LED display 44 and the LCD display 45 can display the charged state of the rechargeable battery 32 through the solar cell 42. That is, the LED display 44 can indicate the charged state of the battery 32 by changing colors of light. Here, the LED display 44 may indicate the charged state of the battery 32 by changing the location of light according to the charged state of the battery 32. Furthermore, the LCD display 45 can indicate the charged state of the rechargeable battery 32 with characters or by percentage. Moreover, the LED display 44 and the LCD display 45 can indicate the charged state of the rechargeable battery 32 at the same time.

Hereinafter, the operation of the nipple overcap having the sterilizer according to the exemplary embodiment of the present invention will be described.

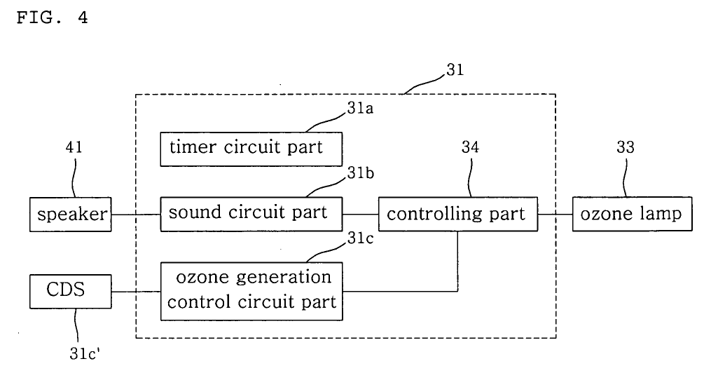

The nursing bottle overcap 20 is fixed on the nipple-fixing cap 12 by using the holding protrusion of the nipple overcap 20 and the holding groove of the nipple-fixing cap 12. Then, when the switch 43 of the substrate case 40 is turned on, as shown in FIG. 4, the controlling part 34 of the electric circuit board 31 respectively transmits signals to the sound circuit part 31b and the ozone lamp 33. The ozone lamp 33 is turned on for sterilizing the nipple 11, and the sound circuit part 31b informs the user of start of sterilization. Here, the user can set a sterilization time period by using the timer circuit part 31a, and control strength of sterilization using the ozone generation control circuit part 31c. The speaker 41 generates informs the user of sterilization progress by a sound. Then, when the sterilization time period set by the timer circuit part 31a is ended, the speaker 41 informs the user of the completion of sterilization by a sound.

Therefore, to let a baby drink milk remaining in the nursing bottle 10 after a predetermined time period after letting the baby drink milk stored in the nursing bottle 10, the user can sterilize and use the nipple 11 mounted on the nursing bottle 10.

In addition, the nipple overcap having the sterilizer according to the exemplary embodiment of the present invention can be coupled with the nursing bottle 10 without using additional cap after letting the baby drink milk after completion of sterilization. Furthermore, the nipple overcap having the sterilization according to the present invention can be carried, and so, the user can conveniently use it out of door at any time.

As described above, the nipple overcap having the sterilizer according to the present invention includes the ozone generator disposed inside the sterilizer detachably mounted on the nipple-fixing cap can sterilize various bacteria parasitic on the nipple. So, the user can sterilize the nipple part within a short period while the user is out or travels together with the baby. Furthermore, the user can continuously sterilize after setting the sterilizing time period, and therefore, does not need to sterilize the nipple part additionally.

While the present invention has been described with reference to the particular illustrative embodiment, it is not to be restricted by the embodiment but only by the appended claims. It is to be appreciated that those skilled in the art can change or modify the embodiment without departing from the scope and spirit of the present invention.

According to the present invention, the nipple overcap having the sterilizer has the following effects.

First, the present invention can sterilize various bacteria since the sterilizer detachably mounted on the nipple-fixing cap includes the ozone generator therein.

Second, the present invention can generate various sounds according to an operation state of the sterilizer since the sterilizer includes the sound generator having a memory storing various sound sources and a speaker. Therefore, the user can easily check the sterilizing state, such as start, progress and completion of sterilization, through various sounds.

Third, the substrate case has the solar cell for absorbing light and converting the light into electric energy, and the electric energy charges the rechargeable battery. Therefore, the present invention does not need additional recharging device, and can be used without exchanging the battery with another battery when the battery is exhausted.

Fourth, the present invention can control the sterilization time period through the timer circuit part mounted on the substrate case for setting the operation time period of the ozone lamp. Furthermore, the present invention can control strength of sterilization through the ozone generation control circuit part.

Fifth, the sterilizer is as small as it can be detachably mounted on the nipple-fixing cap located on the upper portion of the nursing bottle. Therefore, the user can sterilize the nipple part within a short period while the user is out or travels together with the baby. Furthermore, the user can continuously sterilize after setting the sterilizing time period.

Sixth, the present invention can automatically control light of the ozone lamp according to surrounding brightness through the light controlling part for controlling brightness of the ozone lamp.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A nipple overcap having a sterilizer comprising:
a nursing bottle overcap for detachably mounting on a nipple-fixing cap having a nipple and having a through-hole formed at the center thereof, for a part of the upper portion of the nipple;
a substrate case for detachably mounting on the top of the nursing bottle overcap, having a sound generator; and
an ozone generator located between the substrate case and the nursing bottle overcap,
the ozone generator comprising
a rechargeable battery for supplying power to the sound generator and the ozone generator;
an electric circuit board for performing a preset program using the power source supplied from the rechargeable battery; and
an ozone lamp connected to the electric circuit board.

2. The nipple overcap having a sterilizer according to claim 1, wherein the substrate case comprise a solar cell mounted on the upper surface thereof for absorbing light and converting the light into electric energy to charge the rechargeable battery.

3. The nipple overcap having a sterilizer according to claim 1, wherein the sound generator is connected with the rechargeable battery, and comprises:
a memory for storing melodies or sounds; and
a speaker for transmitting melodies or sounds transferred from the memory to the outside,
the speaker being mounted on the substrate case.

4. The nipple overcap having a sterilizer according to claim 2, wherein the substrate case comprises:
a timer circuit part for setting an operation time period of the ozone lamp; and
an ozone generation control circuit part for uniformly controlling generation of ozone by the ozone lamp.

5. The nipple overcap having a sterilizer according to claim 4, wherein the substrate case further comprises an LED display for indicating a progress state.

6. The nipple overcap having a sterilizer according to claim 4, wherein the substrate case further comprises an LCD display for indicating the progress state of sterilization using characters or numbers.

7. The nipple overcap having a sterilizer according to claim 1, wherein the electric circuit board further comprises a light sensitivity controlling part for controlling brightness of the ozone lamp.

8. A nipple overcap comprising:
a nursing bottle overcap for detachably mounting on a nipple-fixing cap;
a substrate case for detachably mounting on the top of the nursing bottle overcap, and having a sound generator;
an ozone generator located between the substrate case and the nursing bottle overcap, the ozone generator comprising a rechargeable battery for supplying power; an electric circuit board for performing a preset program; and an ozone lamp connected to the electric circuit board.

9. The nipple overcap having a sterilizer according to claim 5, wherein the progress state is selected from the group consisting of start, progress, and end of sterilization.
